(19) United States
(12) Patent Application Publication
(10) Pub. No.: US 2024/0408365 A1
(43) Pub. Date: Dec. 12, 2024

(54) MECHANOIONIC CURRENT GENERATOR, A METHOD FOR FABRICATING THE SAME AND A MECHANOIONIC SELF-POWERED DRUG-RELEASING PATCH COMPRISING THE SAME

(71) Applicants: The University of Hong Kong, Hong Kong (HK); Advanced Biomedical Instrumentation Centre Limited, Hong Kong (HK)

(72) Inventors: Lizhi XU, Changsha (CN); Hongzhen LIU, Shenzhen (CN)

(21) Appl. No.: 18/731,433

(22) Filed: Jun. 3, 2024

Related U.S. Application Data
(60) Provisional application No. 63/506,587, filed on Jun. 6, 2023.

(51) Int. Cl. A61M 37/00; H02N 1/04; H02N 2/00; H02N 2/18

(52) U.S. Cl. CPC A61M 37/00; H02N 1/04; H02N 2/181; H02N 2/22; A61M 2037/0007

(57) ABSTRACT

A mechanoionic current generator comprising: a working electrode including an activated carbon cloth; and a counter electrode including a raw carbon cloth and a hydrogel; wherein the hydrogel has a plurality of flexible and asymmetrically shaped structures; and the working electrode has a surface immersed in the hydrogel and provided with a plurality of oxygen-containing functional groups. The ionic current generation mechanism of the current generator is naturally compatible with living organisms and living hydrogels, as exemplified by a self-powered drug delivery patch for wound healing. The current generator provides excellent outputs of current and charge transfer which are advantageous for various biomedical applications.

### CROSS-REFERENCE TO RELATED APPLICATIONS

The present application claims priority from the U.S. Provisional Patent Application No. 63/506,587 filed 6 Jun. 2023, and the disclosure of which is incorporated herein by reference in its entirety

### FIELD OF THE INVENTION

The present invention generally relates to a current generator. More specifically the present invention relates to a mechanoionic current generator based on hydrogel.

### BACKGROUND OF THE INVENTION

Progress towards the intimate integration of electronics into organisms calls for soft and biocompatible mechanoelectric energy converters, which could either be responsive to mechanical stimuli for physiological sensing/monitoring or be able to harvest the rich biomechanical energy for power supply. Conventional electromagnetic generators, despite the large electrical outputs at relatively high frequencies, obviously fall short due to the large mechanical mismatch with the biological systems. The substantially developed triboelectric and piezoelectric nanogenerators, capable of employing various soft materials, are, nevertheless, characterized by high internal impedance and low output current. Given the facts that most biomechanical energy originating from human physiological activities are low-frequency and low-speed, whereas some significant biomedical applications, like muscle electrical stimulation and bone regeneration, require high electrical currents, it is of particular importance to developing soft mechanoelectric devices that can yield high current and high transferred charge at low-frequency and low-speed mechanical energy inputs.

Current generation through mechanoionic mechanisms is a promising approach to meeting these requirements. In analogous to piezoelectric mechanism where the dipoles in dielectric materials are polarized by mechanical pressure, electrolytic materials, such as hydrogels, can also be polarized mechanically due to the deformation-induced unbalanced distribution of cations and anions. The essence is to produce gradient deformation, and therefore, an asymmetric cation/anion distribution at the two electrode/hydrogel interfaces. This asymmetry can be induced by a streaming current of polyelectrolyte hydrogels with single-sign free ions, or produced in neutral hydrogels with cations and anions diffusing at different rates. The latter mechanism was recently demonstrated as a piezoionic effect. Actually, analogous asymmetric ion distribution/diffusion has also been achieved thermally via temperature gradient for thermoelectric generation, and chemically via functional-groups gradient for moisture-induced or water-evaporation-induced electricity generation. Nevertheless, the achievable current output from mechanoionic effect remains at a low level of ˜1 μA, which is not sufficient as a power supply for most practical devices.

### SUMMARY OF THE INVENTION

In accordance with a first aspect of the present invention, a mechanoionic current generator is provided. The mechanoionic current generator comprises: a working electrode including an activated carbon cloth; a counter electrode including a raw carbon cloth and a hydrogel; wherein the hydrogel has a plurality of flexible and asymmetrically shaped structures; and the working electrode has a surface immersed in the hydrogel and provided with a plurality of oxygen-containing functional groups.

In accordance with a second aspect of the present invention, a method for fabricating a mechanoionic current generator comprising a working electrode made of activated carbon cloth, a counter electrode made of carbon cloth and a hydrogel is provided. The method comprises: preparing the working electrode by: cleaning a piece of carbon cloth; oxidizing the cleaned carbon cloth in a two-electrode system containing a (NH₄)₂SO₄ aqueous solution; and reducing the oxidized carbon cloth in a three-electrode system containing a NH₄Cl aqueous solution to form the working electrode; and preparing the counter electrode by: pouring a first PVA solution into a mold to obtain a molded PVA; repeatedly freezing and thawing the molded PVA to obtain a cross-linked hydrogel; immersing a piece of cleaned carbon cloth into a second PVA solution to obtain a PVA-soaked carbon cloth; placing the PVA-soaked carbon cloth onto a back side of the cross-linked hydrogel to obtain a combined structure; repeatedly freezing and thawing the combined structure to obtain a combined electrode; and keeping the combined electrode in an electrolyte solution for 1 day to load the combined electrode with mobile ions to form the counter electrode.

In accordance with a third aspect of the present invention, a mechanoionic self-powered drug-releasing patch is provided. The mechanoionic self-powered drug-releasing patch comprises: a substrate; an electrolyte-containing pad; and a drug-releasing pad disposed between the electrolyte-containing pad and the substrate. The drug-releasing pad comprises: a drug-laden layer having a surface in contact with the electrolyte-containing pad; a printed circuit in contact with the drug-laden layer; and the mechanoionic current generator according to the first aspect of the present invention, wherein the working electrode is electrically connected to the electrolyte-containing pad through the printed circuit; and the counter electrode is electrically connected to drug-laden layer.

In the present invention, engineered structural and chemical asymmetry in hydrogel is optimized in synergy to enhance the net ionic current generation. Mechanoionic current generation amplified by orders of magnitudes is achieved. Under compressive loading, relief structures in the hydrogel intensify deformation-gradient-induced net ion fluxes, which synergize with asymmetric ion-adsorption characteristics of the electrodes and distinct diffusivity of cations and anions in the hydrogel matrix. This engineered mechanoionic process can yield 4 mA (5.5 A m⁻²) of peak current under cyclic compression of 80 kPa applied at 0.1 Hz, with the transferred charge reaching up to 916 mC m⁻² per cycle. The excellent outputs of current and charge transfer are advantageous for biomedical applications. This ionic current generation mechanism is naturally compatible with living organisms and living hydrogels, where ions are the dominant charge/information/energy carriers. On the other hand, hydrogel materials can be further engineered with additional properties, such as bioactivity, biodegradability, bioadhesion, and other characteristics useful in advanced bio-interfaces.

### BRIEF DESCRIPTION OF THE DRAWINGS

Embodiments of the invention are described in more details hereinafter with reference to the drawings, in which:

FIG. 1 shows a structural schematic of a mechanoionic current generator according to one embodiment of the present invention.

FIGS. 2A and 2B illustrate finite element analysis (FEA)-simulated strain before (FIG. 2A) and after (FIG. 2B) applying compression to the hydrogel structure.

FIG. 3 illustrates a working mechanism of the mechanoionic current generator. The strain gradient in the hydrogel layer displaces Cl⁻ with a higher diffusivity than that of L⁺.

FIGS. 4A to 4C illustrate timing profiles of the generated short-circuit current I_{SC}, open-circuit voltage V_{OC} and applied pressure P of the mechanoionic current generator, respectively.

FIGS. 5A and 5B illustrate the voltages and the corresponding current and power density of the mechanoionic current generator for various loads.

FIG. 6 illustrates variation of current outputs when changing the layout of the working and counter electrodes.

FIG. 7 illustrates current outputs of the optimized electrode layout, CC-ACC, with various activation times of the ACC electrodes.

FIG. 8 illustrates the simulated charge redistribution in CC/Li⁺/H₂O system and ACC/Li⁺/H₂O system.

FIG. 9 illustrates calculated adsorption energy of the electrodes to various species of electrode materials.

FIGS. 10A and 10B illustrate contact angles of the steel foil before (91°) and after (32°) plasma treatment, respectively.

FIG. 11A illustrates response currents as functions of applied pressure of various devices using steel foil as the working electrodes; and FIG. 11B illustrates schematic images of corresponding device configurations.

FIG. 12A illustrates response currents as functions of applied pressure and corresponding configuration schematics of various devices using PI/Au as working electrodes; FIG. 12B illustrates schematic images of corresponding device configurations.

FIG. 13 illustrates a schematic illustration of the process of Polyethyleneimine (PEI, a typical cationic polymer) treatment on ACC, altering the ion adsorption characteristics of ACC.

FIG. 14 illustrates distinct current outputs of devices with different treatments of the ACC working electrode.

FIG. 15 illustrates Fourier-transform infrared spectroscopy (FTIR) spectra of ACC at the four stages during the PEI modification process.

FIG. 16 illustrates enlarged FTIR spectrum image, showing that there still exists a small amount of residual PEI on the surface of ACC even after rising for 2 days.

FIG. 17 illustrates peak short circuit current as a function of applied pressure, with the hydrogels soaked with different electrolytes.

FIG. 18 illustrates peak current and charge transferred per cycle as functions of the recovery time between cycles.

FIG. 19 illustrates an output current under various recovery times.

FIG. 20 illustrates peak current and charge transferred per cycle as functions of the strain rate.

FIG. 21 illustrates output current under various strain rates.

FIG. 22 illustrates a charging profile of a 2 F capacitor charged by the hydrogel-based current generator.

FIG. 23 illustrates a voltage of a 2 F capacitor during charging the 2 F capacitor by an electrochemical workstation.

FIG. 24 illustrate an demonstration setup using the mechanoionic current generator as a power supply to harvest mechanical energy and drive electronics.

FIG. 25 illustrates a current output over 7500 cycles of 5 kPa compression, showing durability and stability of the device.

FIG. 26 illustrates pressure-distance curve of the 1^{st} and 500^{th} compression-separation cycle.

FIG. 27A illustrates pressure-response characteristics of the large-scale device with the device area increased to 29 cm²; FIG. 27B illustrates current and voltage when the large-scale device is loading different resistance; FIG. 27C illustrates power density of the large-scale device as a function of external resistance.

FIG. 28 illustrates current signals when loading 122Ω resistance under low and high humidity.

FIG. 29 illustrates a recycling process of the CC-PVA electrode.

FIG. 30 illustrates a comparison of the mechanoionic current generator provided by the present invention with state-of-the-art in the domains of internal resistance, current density, softness, transferred charge, and density.

FIG. 31 illustrates current outputs of this hydrogel-based current generator under various frequencies of mechanical input, as compared with those of TENG and EMG.

FIG. 32 illustrates a schematic of drug-loading process with electrodeposition.

FIG. 33A illustrates SEM images of drug-releasing patch in scale of 1:50 μm; and FIG. 33B illustrates SEM images of drug-releasing patch in scale of 1:5 μm.

FIG. 34 illustrates a schematic of the electro-responsive drug release in vitro.

FIG. 35 illustrates a standard calibration curve of cefazolin sodium.

FIG. 36 illustrates a vitro demonstration of electrically controlled drug release with “on-off” cycles driven by the hydrogel-based current generator. The “on” state represents tapping the device to provide output current.

FIG. 37 illustrates schematic (upper) and photography (bottom) of the self-powered drug-releasing patch (SDP).

FIG. 38A illustrates a schematic showing application of the SDP on the dorsal flank side of a mouse; and FIG. 38B illustrates a schematic image to show the work mechanism of the developed SDP.

FIG. 39 illustrates equivalent circuit of the SDP.

FIG. 40 illustrates the relative area of wounds as a function of healing time. On the last day, the wound area covered by the normal patch and SDP was reduced to 32% and 11% of the initial wound area, respectively.

FIG. 41 illustrates photographs of the wound during the healing process with a standard patch (upper) and the SDP (lower).

### DETAILED DESCRIPTION

In the following description, a mechanoionic current generator and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

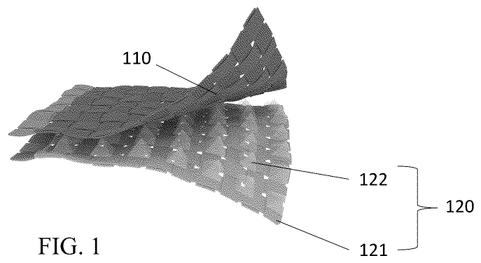

FIG. 1 shows a structural schematic of a mechanoionic current generator according to one embodiment of the present invention. As shown, the mechanoionic current generator includes a working electrode 110 and a counter electrode 120.

The working electrode 110 is made of activated carbon cloth (ACC). The counter electrode 120 is made of raw carbon cloth (CC) 121 and a hydrogel 122. The hydrogel 122 has a plurality of flexible and asymmetrically shaped structures. A bottom surface of the working electrode 110 immersed in the hydrogel 122 and provided with a plurality of oxygen-containing functional groups. The plurality of oxygen-containing functional groups is configured to face toward tip portions of the plurality of flexible and asymmetrically shaped structures of the hydrogel 122. Each of the flexible and asymmetrically shaped structures has a pyramid-like shape.

The ACC working electrode 101 may be fabricated by: cleaning a piece of CC with acetone, isopropyl alcohol (IPA), and de-ionized water (DI water) before activation; and subjecting the CC to an electrochemical oxidative process carried out in a two-electrode system in 0.1 M (NH₄)₂SO₄ aqueous solution using a Pt electrode as the counter electrode and applying a voltage of 10 V for 15 min. the oxidized CC is subjected to an electrochemical reduction process carried out in a three-electrode system in 1 M NH₄Cl aqueous solution with the Pt and Ag/AgCl electrodes serving as the counter and reference electrodes, respectively. The oxidized carbon cloth is reduced at −1.2 V for 30 min. After cleaning and drying, the as-fabricated ACC was collected and used as the working electrode.

The CC-PVA counter electrode may be fabricated by: pouring 15 wt % PVA solution into a 3D-printed PTFE mold, freezing the molded PVA at −18° C. for 8 h, and thawing the frozen PVA at 25° C. for 3 h; and repeating the afore-said freezing and thawing steps four times to obtain a physical cross-linked hydrogel. Then a piece of cleaned CC is immersed into 10 wt % PVA solution and placed onto the back side of the cross-linked PVA hydrogel. After repeating the freezing-thawing process twice, the CC-PVA electrode was kept in an electrolyte solution (4 M LiCl) for 1 day to load the CC-PVA electrode with mobile ions L⁺ and Cl⁻.

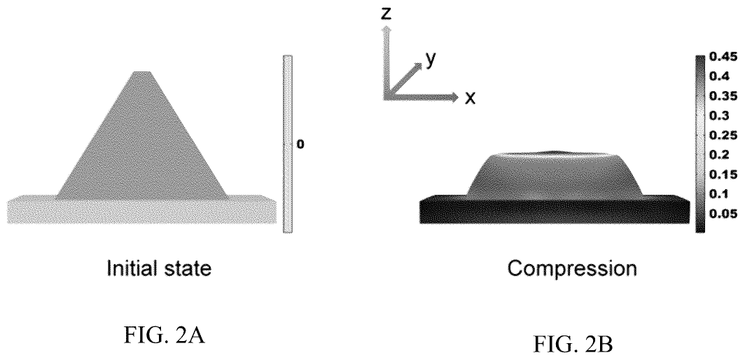

FIGS. 2A and 2B illustrate finite element analysis (FEA)-simulated strain before (FIG. 2A) and after (FIG. 2B) applying compression to the hydrogel structure. The sectional area of the hydrogel pyramid changes with the height, resulting in a high strain gradient under normal compression. This strain gradient displaces the water from the region with large compressive strain to that with smaller strain, and then the resulting gradient of the ion concentration leads to the cation/anion diffusion along the strain gradient.

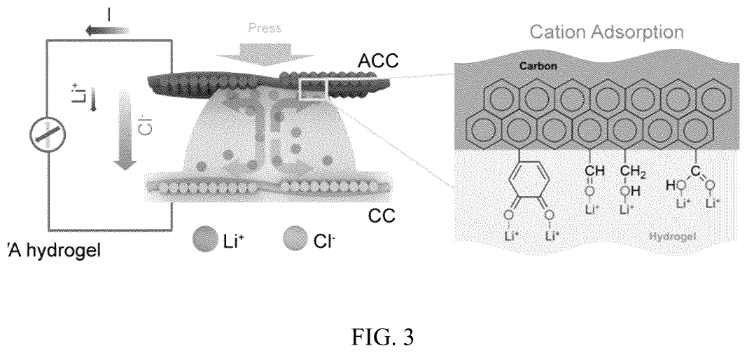

FIG. 3 illustrates a working mechanism of the mechanoionic current generator. The strain gradient in the hydrogel layer displaces Cl⁻ (blue) with a higher diffusivity than that of Li⁺ (red). On the other hand, the ACC in the working electrode possesses stronger adsorption to Li⁺ cations than Cl-anions, leading to an enhanced separation of charges. The enlarged figure shows the electrode-ion interaction at the ACC/hydrogel interface. Due to the different diffusivity of cations and anions, a net ionic current will be generated inside the hydrogel and, therefore, an electric current through the external circuit.

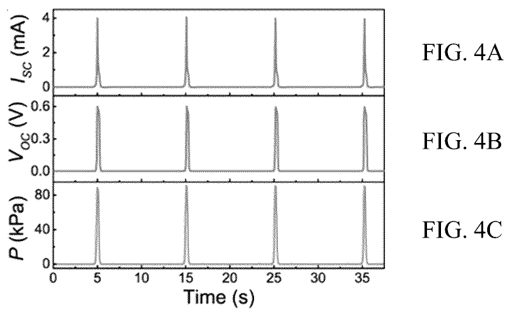

FIGS. 4A to 4C illustrate timing profiles of the generated short-circuit current I_{SC}, open-circuit voltage V_{OC} and applied pressure P of the mechanoionic current generator, respectively. As shown, the current generator outputs a large current pulse under compressive deformation. The current can be as high as 4 mA (5.5 A m⁻²) under 80 kPa pressure, with the transferred charge being 916 mC m⁻², three to five orders of magnitude larger than existing devices (Table 1), reaching the threshold for direct electrical stimulation for muscles.

TABLE 1
Comparison with other mechanical-electric energy converts.

| | | | | | | | Charging | Charge |
|---|---|---|---|---|---|---|---|---|
| | | Current | Power | Resistance | Loading rate | | rate | transfer |
| Type | Output | mA cm⁻² | W m⁻² | Ω | Hz: cm s⁻¹ | Form factor | mC s⁻¹ | mC m⁻² |
| PENG | AC | 0.00064 | 0.113 | 6000000 | 2 [symbol] | Soft | 0.0294 | 0.147 |
| | AC | 0.00071 | 0.0653 | 1E+07 | 1 [symbol] | Soft | 1.07 | 3.6 |
| | AC | 0.00073 | 0.0605 | 7E+07 | 2 [symbol] | Soft | 0.0396 | 0.495 |
| TENG | AC | — | 0.001 | 4000000 | 5: 100 | Soft | — | — |
| | AC | 0.0017 | 0.328 | 7000000 | 1.5: 20 | Soft | 0.0619 | 0.0516 |
| | AC | 0.00179 | 1.59 | 1E+07 | 2 [symbol] | Soft | — | — |
| | AC | 0.0765 | 142.5 | 800000 | 4 [symbol] | Flexible | 15.7 | 3.53 |
| | AC | 0.0025 | 10.5 | 200000 | [symbol] | Flexible | 1 | 0.05 |
| SDC-TENG | DC | 0.006 | 0.00015 | 620 | #1: 50 | Hard | — | — |
| | DC | 0.25 | 11.85 | 160000 | #2: 50 | Hard | 96.5 | 136.3 |
| MEG | AC | 4.27 | 20.17 | 30 | 2 [symbol] | Soft, Weight | 3.3 | 8.25 |
| Piezonionic | DC | 0.011 | 0.00085 | 500 | 0.1-1 [symbol] | Soft | — | 800 |
| This work | DC | 0.55 | 0.475 | 122 | 0.05-1: 0.2-3.3 | Soft, Light | 159.7 | 916 |

# driving method: pressing and sliding.
#1: pressure 50 kPa:
#2: pressure 30 kPa [symbol]
indicates data missing or illegible when filed

The transferred charge amount (Q) was calculated by

$$Q = \int Idt \qquad ( 1 )$$

where I is the current and t is the time. Before testing, the surface of the hydrogel and the counter electrode were wiped dry.

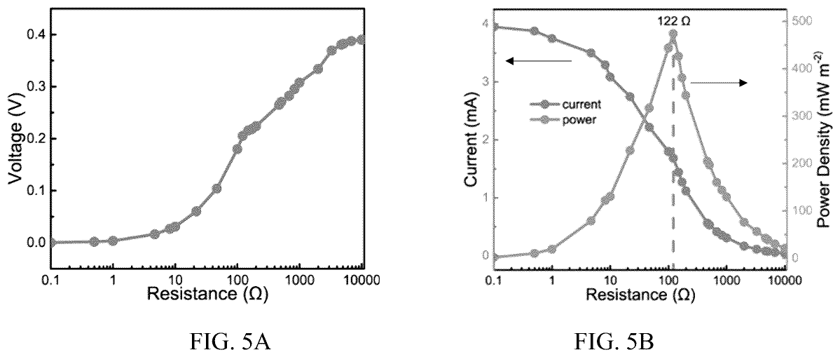

FIGS. 5A and 5B illustrate the voltages and the corresponding current and power density of the mechanoionic current generator for various loads.

The power density (P) was calculated by

$$P = {I}^{2} / ( R \times S ) \qquad ( 2 )$$

with I, R, S being the recorded current, the resistance of loading, and the area of the device, respectively.

As shown in FIGS. 5A and 5B, the maximum peak power density of 475 mW m⁻² is achieved with an external load of 122Ω, suggesting that the internal resistance of the hydrogel-based current generator is less than one thousandth of triboelectric or piezoelectric nanogenerators (TENG or PENG). Compared to the recently reported generator based on piezoionic effects, the provided hydrogel-based mechanoionic current generator also shows advantages in the current (4 mA verse 1 μA) and power (475 mW m⁻² verse 0.85 μW cm⁻³) outputs.

By leveraging the asymmetric surface functionalization of the electrodes, the mechanoionic electricity generation can be significantly improved. Specifically, high current output is achieved by making use of the synergetic effect between the differential ion-adsorption properties of electrodes and the differential ion diffusion in the hydrogel matrix. That is, the difference in diffusivity of Cl-anions and Li cations can be enhanced in PVA hydrogels. The configuration of Li⁺-anchoring ACC electrode interfacing with the high-strain region can further reduce the diffusion flux of Li⁺, in turn to enhance the generation of ionic current.

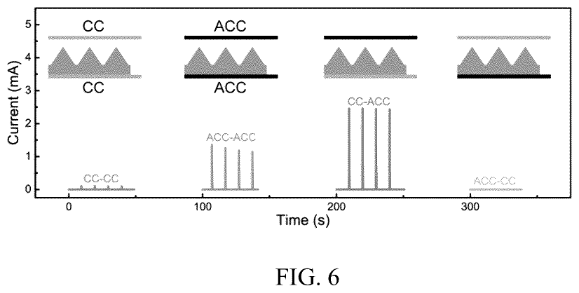

A set of control experiments well demonstrate these effects. As illustrate in FIG. 6, two generators with symmetric pairs of electrodes (using two CC or ACC electrodes) were tested for comparison, both showing electrical current generation but a much smaller peak value. On the other hand, little generation of current was observed if the ACC working electrode and CC counter electrode were reversed, since the ion-anchoring effects were flipped and counteracted with the differential diffusion of cations and anions.

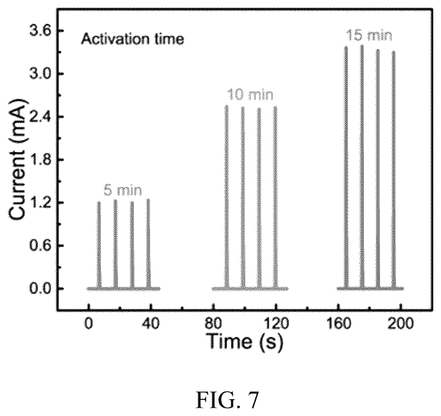

FIG. 7 illustrates current outputs of the optimized electrode layout, CC-ACC, with various activation times of the ACC electrodes. As shown, the current increased with the time duration of electrochemical oxidation of the ACC working electrode.

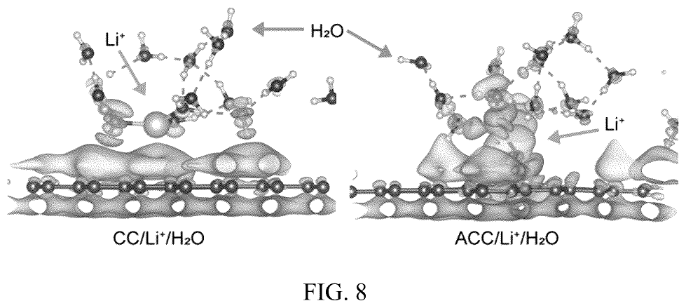

To confirm the role of oxygen-containing functional groups for the anchoring of Li⁺, it is calculated the charge redistribution when CC and ACC are in contact with the electrolyte by density functional theory (DFT). FIG. 8 illustrates the simulated charge redistribution in CC/Li⁺/H₂O system and ACC/Li⁺/H₂O system. The charge redistribution process mainly occurs between H₂O molecules and CC for CC/Li⁺/H₂O system; whereas, for ACC/Li⁺/H₂O system, it is evident that Li⁺ gains electrons while ACC loses electrons. Hence the adsorption energy of LiCl on ACC (1.2 cV) is higher than that on CC (0.18 cV).

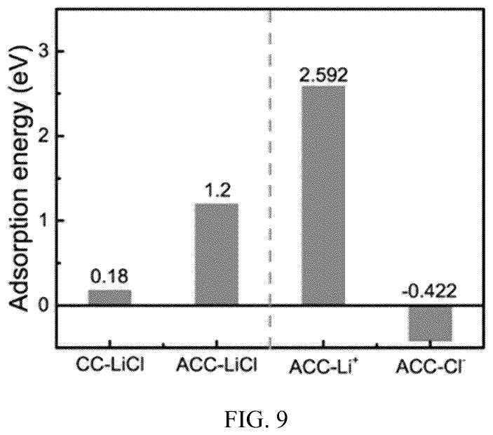

FIG. 9 illustrates calculated adsorption energy of the electrodes to various species of electrode materials. For ACC, the adsorption energy of L⁺ and Cl⁻ are calculated to be 2.515 cV and −0.384 eV, respectively. These results indicate that when ACC is in contact with the hydrogel, Li⁺ cations tend to accumulate at the ACC-hydrogel interface, while Cl⁻ anions tend to stay away from the interface.

In some embodiments, the top working electrode may be changed from ACC to gold (Au) film or stainless-steel foil. Oxygen plasma treatment was applied to these metal electrodes for introducing oxygen-containing groups on the surface.

In a typical fabrication process of PI/Au electrode, a piece of PI film (thickness: 50 μm) was cleaned with acetone, IPA, and DI water. Then a bilayer of chromium (Cr, 5 nm) and gold (Au, 200 nm) was sputtered (Denton desktop pro sputter system) on the PI film.

In a typical fabrication process of the steel electrode, a piece of stainless-steel foil (50 μm) was cleaned with sandpaper (7000 mesh), acetone, IPA, and DI water.

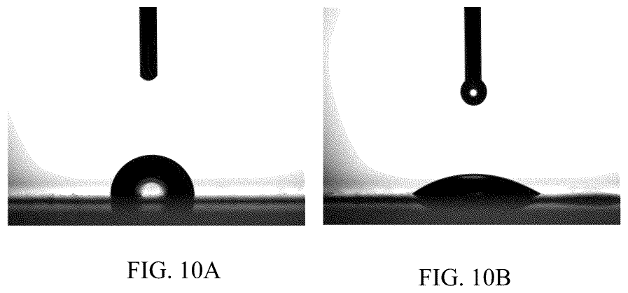

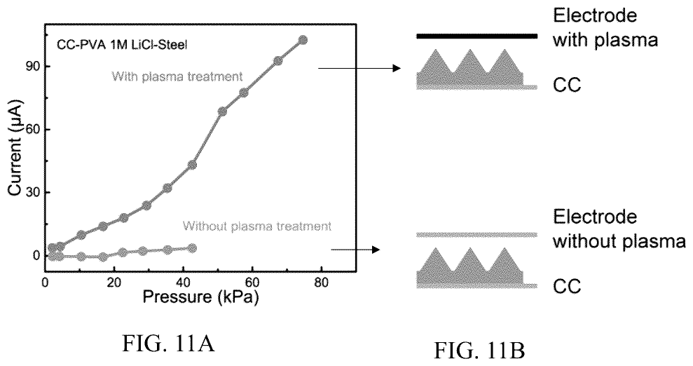

The oxygen plasma treatment (40 sccm oxygen flow, 100 W, and 30 s) was carried out by an RIE etcher (Tailong Electronics). FIGS. 10A and 10B illustrate contact angles of the steel foil before (91°) and after (32°) plasma treatment, respectively. The lower contact angle after plasma treatment indicates the introduction of oxygen-containing functional groups.

FIG. 11A illustrates response currents as functions of applied pressure various devices using steel foil as the working electrodes. FIG. 11B illustrates schematic images of corresponding device configurations. After plasma treatment, the output current increased significantly in both cases. This result is in good agreement with the results mentioned in FIG. 6 when ACC and CC were used as the working electrode, showing the importance of oxygen-containing functional groups.

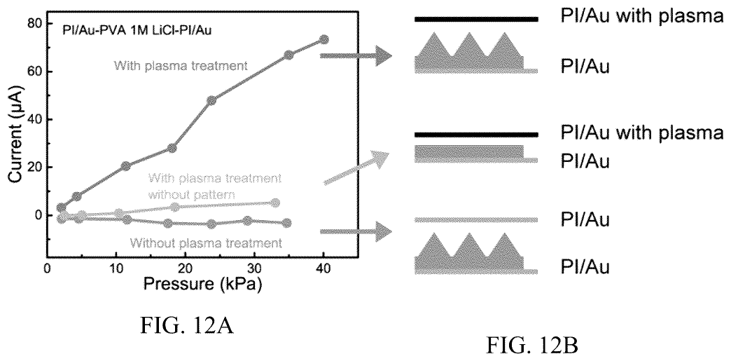

FIG. 12A illustrates response currents as functions of applied pressure and corresponding configuration schematics of various devices using PI/Au as working electrodes. FIG. 12B illustrates schematic images of corresponding device configurations. In FIGS. 12A and 12B, the tremendous difference in response currents before and after plasma treatment also shows that oxygen-containing functional groups are vitally important. Meanwhile, the difference between the yellow and red lines proves that the engineered pyramid structure also plays a critical role in improving the current. The PI/Au film is chosen because it is very flat and clean, which can significantly reduce the influence of undesired surface deformation of the hydrogel. The value of the blue line is below zero. A possible reason is that when using carbon electrodes, cations may be preferentially adsorbed over anions due to the cation-π interactions, which is in contrast to the metal-electrolyte interface, where the cation-π interactions are absent and anion adsorption may dominate. Thus, the direction of the current is reversed.

Therefore, it is evident that surface functionalization with abundant oxygen-containing groups of the working electrode can greatly enhance the current output due to the anchoring effect for Li⁺. The selective adsorption induces the separation of anions and cations at the surface between the activated carbon cloth and the hydrogel. Increasing the asymmetry of the diffusion flux can effectively increase efficiency. Moreover, improving the selective ion adsorption by matching the electrolyte and electrodes can further improve the response currents.

Another set of control experiments further demonstrates the importance of selective ion adsorption on mechanoionic current generation. Specifically, the surface functionalization of the ACC working electrode is treated with Polyethyleneimine (PEI, a typical cationic polymer) such that it is changed to be capable of anion adsorption, which is opposite to its optimal design.

Before measurement, a piece of ACC is cleaned and then immersed into 25 wt % polyethyleneimine (PEI) solution (600 MW, Aladdin Company). PEI with positively charged groups was adsorbed onto the surface of ACC during the 24 h immersion. Then the PEI-modified ACC, named ACC-PEI, was completed by drying it in an oven at 85° C.

After measurement, this PEI-modified ACC electrode was rinsed with DI water for 2 min and dried again. This step aims to reduce the content of PEI. After that, the electrode was tested again. Afterward, the ACC was immersed in DI water for 2 days at 85° C. to remove most of the adsorbed PEI. The as-fabricated ACC was tested after drying.

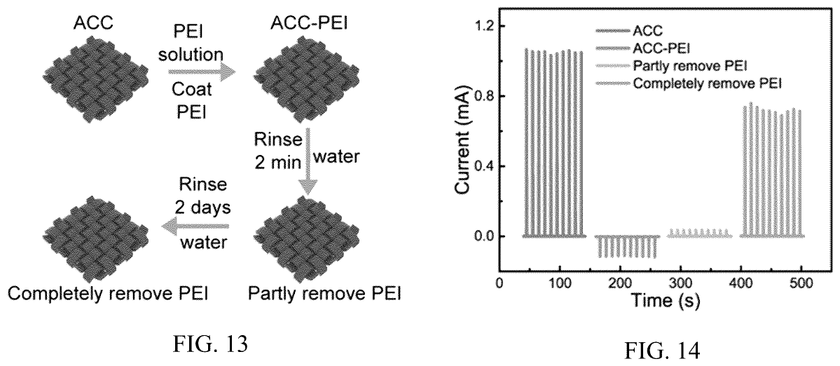

FIG. 13 illustrates a schematic illustration of the process of Polyethyleneimine (PEI, a typical cationic polymer) treatment on ACC, altering the ion adsorption characteristics of ACC. FIG. 14 illustrates distinct current outputs of devices with different treatments of the ACC working electrode.

An ACC-PEI electrode is prepared by applying PEI on the surface of the ACC by immersing the ACC into a 25 wt % PEI solution and drying it in an oven, it is observed that the direction of current was reversed when changing the working electrode from ACC to ACC-PEI. When using ACC as the working electrode and 1 M LiCl hydrogel as the electrolyte in the optimum configuration, a positive peak current of 1.051 mA was obtained at 12 kPa pressure. However, the output changed to an opposite current of −0.111 mA when using ACC-PEI as the working electrode. Subsequently, the ACC-PEI was rinsed with DI water for 2 min to partly remove the PEI and dried again to reduce the PEI content. The response current returned positive with a small value of 0.0349 mA. Further reducing the PEI content by rinsing ACC-PEI for 2 days in DI water recovered the response current back to 0.722 mA. The small amount of PEI residue on the ACC was responsible for the slight decrease in the current compared with the initial value.

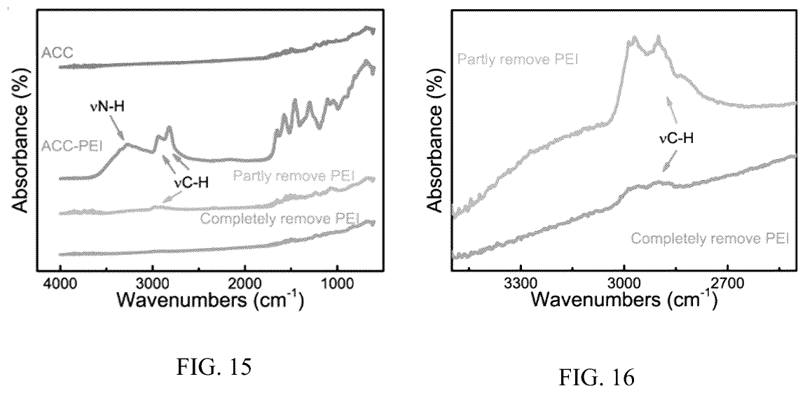

The variation of PEI content in each step was characterized by Fourier-transform infrared spectroscopy (FTIR) spectroscopy. FIG. 15 illustrates FTIR spectra of ACC at the four stages during the PEI modification process. FIG. 16 illustrates enlarged FTIR spectrum image, showing that there still exists a small amount of residual PEI on the surface of ACC even after rinsing for 2 days. As a typical cationic polymer with a strong positive surface charge, PEI is prone to adsorbing negative ions and repelling positive ions. Due to the abundant negatively charged groups, PEI can be adsorbed on the surface of ACC. In this way, the surface charge status is changed from negative to positive, endowing it with the ability to absorb anions.

Peaks located at 3250 cm⁻¹ for v(N—H) and 2925 cm⁻¹ and 2830 cm⁻¹ for v(C—H) were identified as the characteristic peaks of PEI. The changes in the intensity of these peaks indicate the successful modification and the gradual removal of PEI. These results indicate that the anion adsorption superiority of the ACC-PEI working electrode can overwhelm the strain gradient-induced net anion flux, and lead to the reversed sign of net cation flux. In this regard, the mechanoionic effect can be well tuned by appropriately designing asymmetric chemical functionalization of the electrodes, which is convenient for diverse device applications.

The performance of these mechanoionic hydrogel-based current generators is further characterized in the context of practical applications. The optimized electrode layout (ACC as the working electrode and CC as the counter electrode) was used for all the tests thereafter.

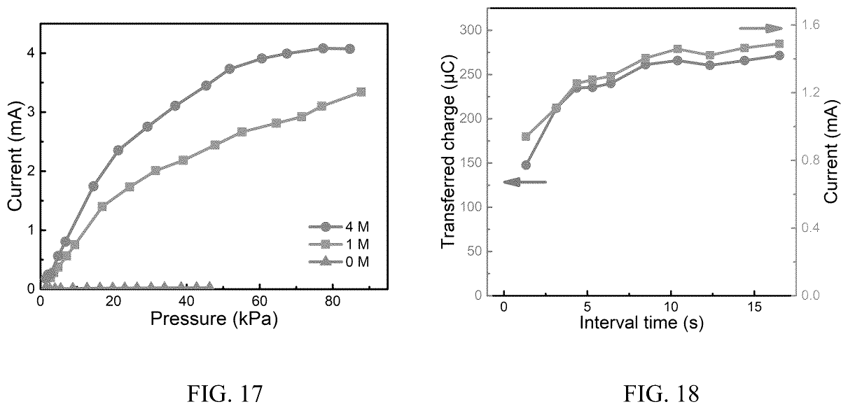

The influences of ionic concentration and applied pressure on the current output were first studied. FIG. 17 illustrates peak short circuit current as a function of applied pressure, with the hydrogels soaked with different electrolytes: DI water (gray), 1 M LiCl (blue) and 4 M LiCl (red). No current signal was observed under any pressure when DI water-based hydrogel served as the electrolyte. When using hydrogel with 1 M LiCl, the response current went up apparently from 0 mA to 3.3 mA with increasing pressure from 0 kPa to 88 kPa. Higher current output was obtained using hydrogel with 4 M LiCl. The current increased with pressure but reached a saturation of about 4 mA at around 60 kPa.

Next, it is studied the influences of recovery time between cycles and strain rates on the current generation. With the loading rate fixed at 16 mm s⁻¹ (strain rate: 2 s⁻¹), the response current increases with the extension of the recovery time from 1 s to 8 s (frequency: 1 Hz to 0.125 Hz) and keeps constant when recovery time exceeds 8 s.

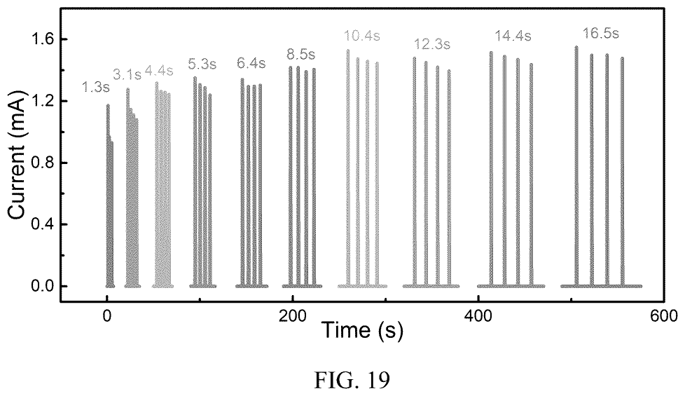

FIG. 18 illustrates peak current and charge transferred per cycle as functions of the recovery time between cycles. FIG. 19 illustrates an output current under various recovery times. Longer recovery time (lower frequency) generates more stable and higher currents, which may be attributed to the transient distribution of ions.

This phenomenon can be explained as that ions cannot restore the initial uniform distribution within the short recovery time, causing a gradual decrease in current at higher frequencies. Transferred charge per pressing-releasing cycle presents the same trend. Even if the recovery time was shortened to 1 s, the amount of transferred charge was up to 180 μC per cycle.

Higher strain rate results in a higher peak current. After removing the external pressure, it takes a certain time for ions to return to the initial distribution state. Higher strain rates generate more significant deformation of the hydrogel in a short time, leading to a higher concentration gradient and, thus, higher output current. Meanwhile, higher strain rates shorten the period for ion redistribution, and lead to lower amount of transferred charge per cycle.

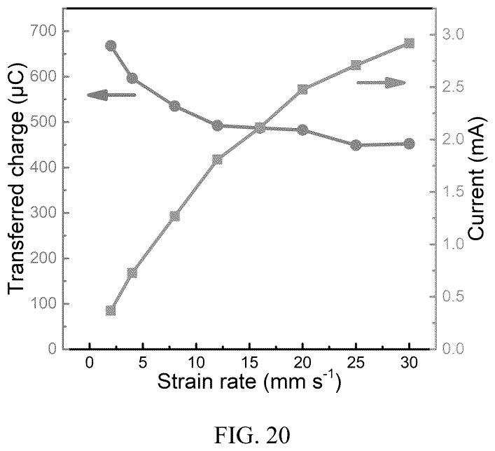

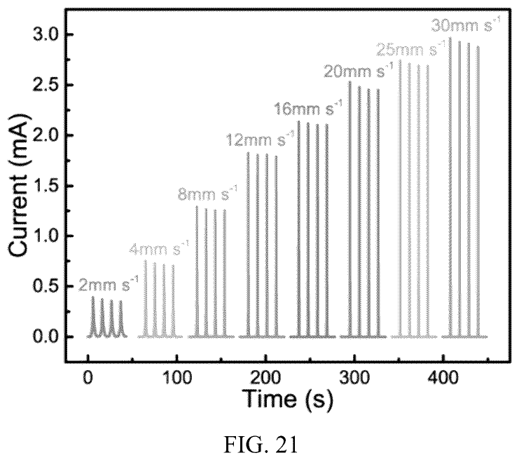

FIG. 20 illustrates peak current and charge transferred per cycle as functions of the strain rate. FIG. 21 illustrates output current under various strain rates. With recovery time fixed at 10.4 s, the strain rate was changed from 2 mm s⁻¹ to 30 mm s⁻¹, with the corresponding peak current increasing from 0.4 mA to 2.9 mA. A higher strain rate leads to a higher gradient in ion concentration due to limited response time for ion redistribution, hence enhancing the differential ionic diffusion flux. On the other hand, a higher strain rate shortens the duration of the current output, reducing the transferred charge per cycle. At a 2 mm s⁻¹ strain rate, the transferred charge reached 668 μC (916 mC m⁻²) per cycle, which outperforms the reported mechanoelectric energy converters. Benefited from the high capability in charge transfer, the hydrogel-based current generator can charge capacitors quickly.

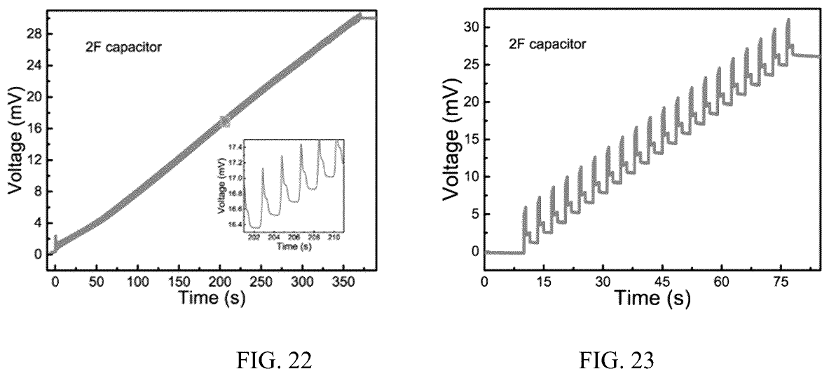

FIG. 22 illustrates a charging profile of a 2 F capacitor charged by the hydrogel-based current generator. As shown in FIG. 22, the 2 F capacitor can be charged to 29 mV in 350 s, with the charging rate being as high as 166 μC s⁻¹.

FIG. 23 illustrates a voltage of a 2 F capacitor during charging the 2 F capacitor by an electrochemical workstation. In order to simulate the current pulse generated by the device, charging parameters were set as followings: 4 mA for 0.5 s, 1 mA for 1 s, and 0 mA for 2 s. Voltage fluctuation is observed, as mentioned in FIG. 22, which may be attributed to the influence of the internal resistance of the capacitor.

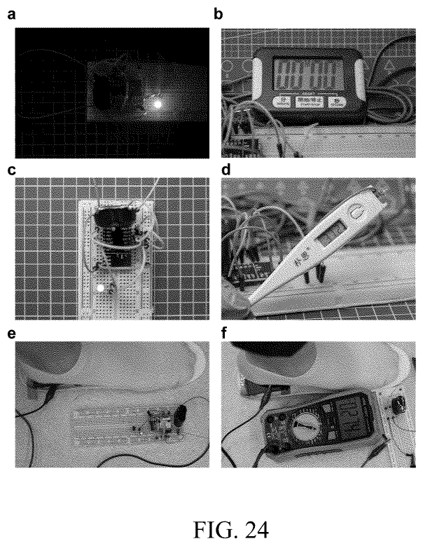

The fluctuation of current during the cycle is similar to that when charging the capacitor using a potentiometer (FIG. 23). Together with a power management system, the hydrogel-based current generator can collect mechanical energy and charge a 2 F capacitor, which then powered a LED, a thermometer, and a liquid crystal display (FIG. 24). An insole integrated with the hydrogel-based current generator was designed to harvest mechanical energy from walking and store the charge in a capacitor (FIG. 24).

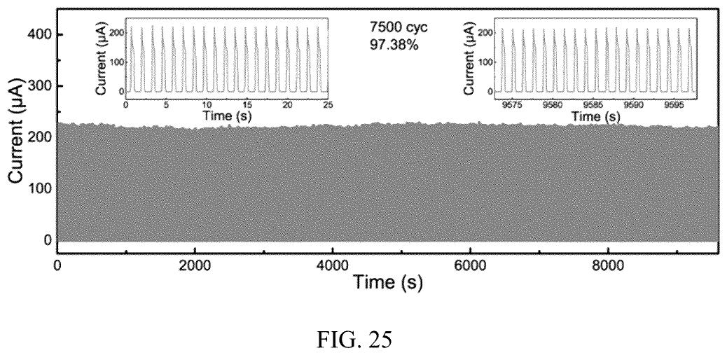

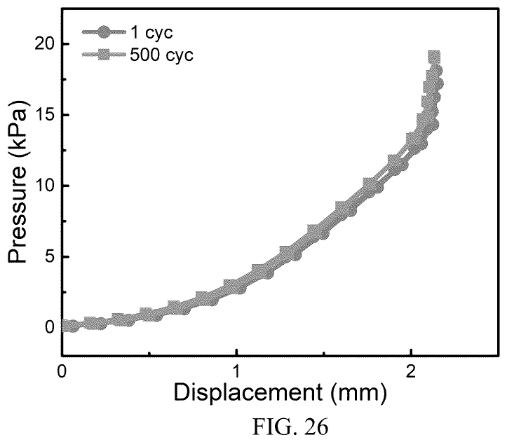

Furthermore, the hydrogel-based current generator has outstanding durability, showing no significant degradation of output over 7,500 compression cycles (FIG. 25). This durability originates from the excellent elasticity of the device during loading cycles. FIG. 26 illustrates pressure-distance curve of the 1^{st} and 500^{th} compression-separation cycle. Highly overlapping curves indicate the mechanical stability of the device.

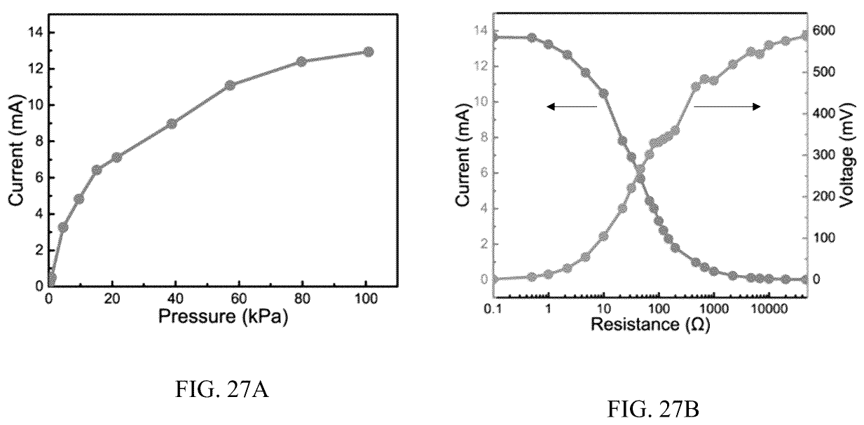

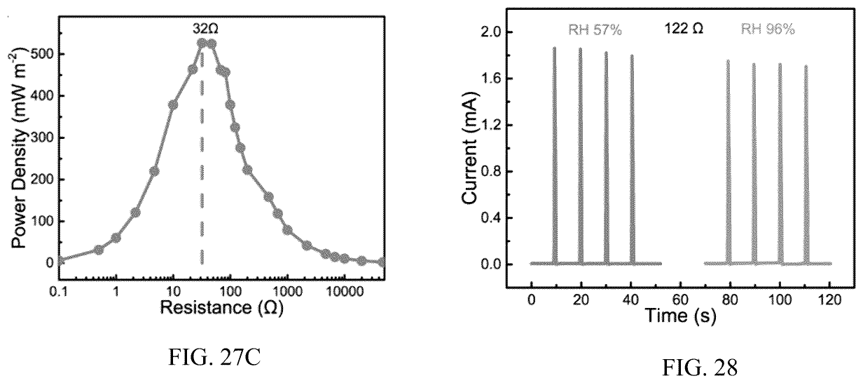

The device can also be scaled up. FIG. 27A illustrates pressure-response characteristics of the large-scale device with the device area increased to 29 cm². A peak current approaching 13 mA is achieved. FIG. 27B illustrates current and voltage when the large-scale device is loading different resistance. FIG. 27C illustrates power density of the large-scale device as a function of external resistance. The change in the area can explain the decrease in internal resistance.

In FIGS. 27A to 27C, the change in the area can explain the decrease in internal resistance. Since the thickness and composition of this large-scale device are similar to the original device (with a device area of 7.29 cm²), it is reasonable to assume the same resistivity for both. Thus,

$${R}_{large} = {R}_{small} \times \frac{{s}_{small}}{{s}_{large}} = 122 \times \frac{7.29}{29} Ω = 31 Ω ,$$

which is close to the measured value of 32Ω.

FIG. 28 illustrates current signals when loading 122Ω resistance under low and high humidity, showing that the electrical output is not sensitive to the external humidity. It can be seen that the device can keep stable output even in a high-humidity environment, which would be otherwise difficult for triboelectric nanogenerators.

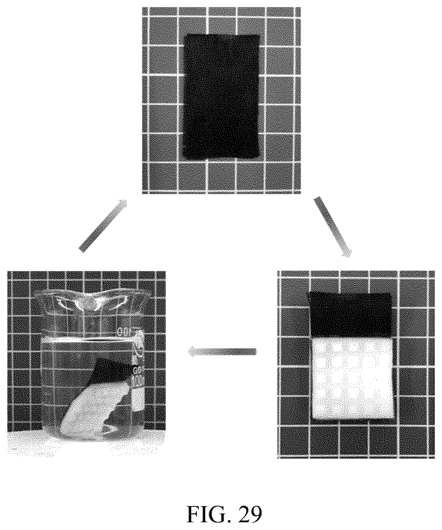

In addition, due to the physical cross-linking of PVA, the hydrogel part can be recycled for disused devices. FIG. 29 illustrates a recycling process of the CC-PVA electrode. Putting the counter electrode into hot water can dissolve the physical-crosslinking PVA hydrogel, recycling the carbon cloth and PVA.

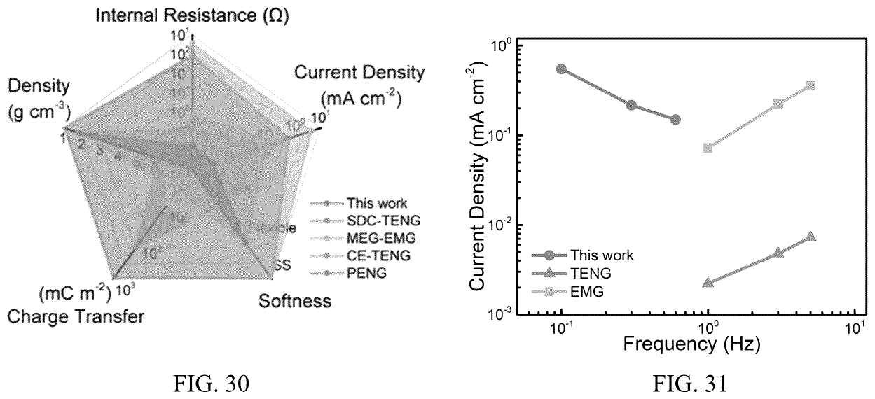

Compared with state-of-the-art mechanoelectric energy converters, including semiconductor direct-current triboelectric nanogenerator (SDC-TENG), magnetoelastic generator (MEG), TENG, and PENG, this mechanoionic hydrogel-based current generator is advantageous in terms of internal resistance, current density, transferred charge density, and device softness (FIG. 30 and Table 1).

FIG. 31 illustrates current outputs of this hydrogel-based current generator under various frequencies of mechanical input, as compared with those of TENG and EMG. The test parameters are listed in Table 2. In contrast to EMG and TENG, the hydrogel-based current generator is especially suitable for harvesting very-low-frequency mechanical energy, and the current output increases with decreasing frequency. Furthermore, considering the mechanical softness and biocompatibility of the hydrogel-based current generator, it is promising for applications in soft robotics and biomedical devices.

TABLE 2
Parameters used in tests and demonstrations

| | | | | Recovery | | |
|---|---|---|---|---|---|---|
| | | Electrolyte | Electrodes | time | Strain rate | Pressure |
| FIG. 1 | d, e | 4M LiCl | CC-ACC | 10.4 s | 16 mm s⁻¹ | 80 kPa |
| FIG. 2 | a | 1M LiCl | — | 10.4 s | 16 mm s⁻¹ | 47 kPa |
| | b | 1M LiCl | CC-ACC | 10.4 s | 16 mm s⁻¹ | 80 kPa |
| | f | 1M LiCl | — | 10.4 s | 16 mm s⁻¹ | 12 kPa |
| FIG. 3 | a | — | CC-ACC | 10.4 s | 16 mm s⁻¹ | — |
| | b | 1M LiCl | CC-ACC | — | 16 mm s⁻¹ | 17 kPa |
| | c | 1M LiCl | CC-ACC | 10.4 s | — | 34 kPa |
| | d | 4M LiCl | CC-ACC | 1.87 s | 16 mm s⁻¹ | 75 kPa |
| | e | 4M LiCl | CC-ACC | 1.28 s | 16 mm s⁻¹ | 5 kPa |
| FIG. 4 | b | 4M LiCl | CC-ACC | — | 16 mm s⁻¹ | 80 kPa |
| | c | 4M LiCl | CC-ACC | hand | hand | hand |
| | | | | tapping | tapping | tapping |
| | d | 4M LiCl | CC-ACC | — | — | — |
| Default | | 4M LiCl | CC-ACC | 10.4 s | 16 mm s⁻¹ | 80 kPa |

—: variable or not applicable.
Recovery time: Time interval between pressure peaks in two cycles.

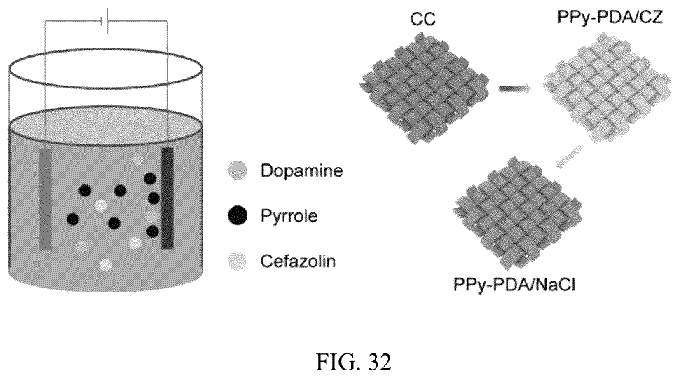

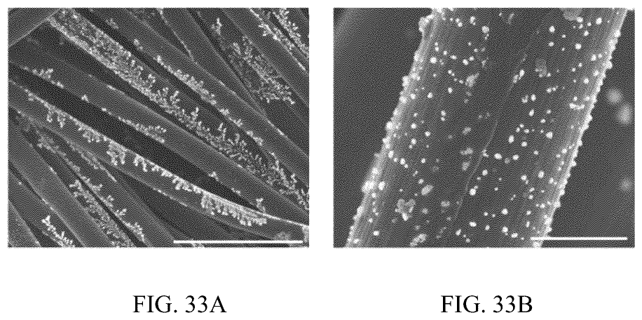

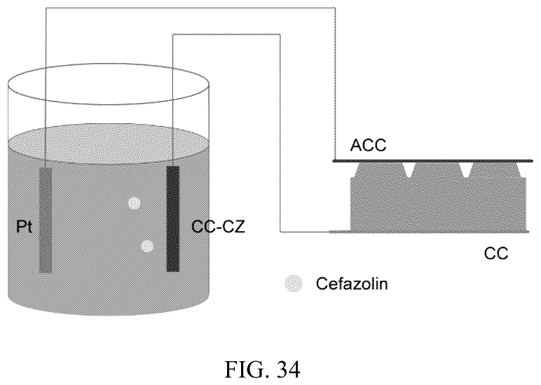

To demonstrate its utility in biomedical applications, the hydrogel-based current generator is used as the power source for an integrated drug-releasing system. FIG. 32 illustrates a schematic of drug-loading process with electrodeposition. A polypyrrole (PPy) layer doped with dopamine and an antibiotic (cefazolin sodium) was electrodeposited onto a carbon cloth (FIGS. 33A and 33B). As illustrated in FIG. 34, a two-electrode system was employed, in which the drug-laden carbon cloth was the working electrode, a Pt foil was the counter electrode, and 10 ml PBS buffer was the electrolyte. Under periodic tapping, the generated current transforms PPy from the oxidized state to the reduced state, releasing the negatively charged cefazolin from PPy to PBS solution.

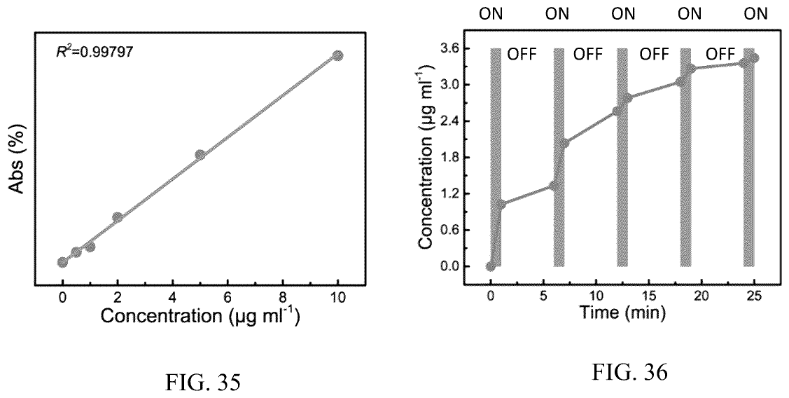

The concentration of the drug was calibrated by UV-VIS spectroscopy a series of PBS buffers containing different concentrations of the drug (cefazolin sodium). The UV-Vis spectrometer defined the relationship between the absorbance and the drug concentration at 272 nm, which is the characteristic absorbance band of cefazolin sodium. Therefore, a standard calibration curve of cefazolin sodium (FIG. 35) is plotted, with R²=0.99797.

Due to the high current output of the generator, the drug release rate when tapping the device is much higher than that without tapping (FIG. 36), suggesting a design for electromechanically controlled release of drugs.

The integrated drug-releasing system is further integrated with an Ag/AgCl electrode to form a drug-releasing pad. In other words, the drug-releasing pad includes a three-electrode system constructed with a piece of cleaned CC, a Pt electrode, and an Ag/AgCl electrode, serving as the working electrode, the counter electrode, and the reference electrode, respectively. A constant voltage of 0.8 V was applied to the system for 300 s with the electrolyte solution containing 0.1 M pyrrole, 0.01 M dopamine hydrochloride, and 0.01 M cefazolin sodium. After that, a layer of chloride-doped polypyrrole was deposited via electrodeposition (0.8 V, 60 s) to slow down the natural release of the drug (the electrolyte contains 0.1 M pyrrole, 0.01 M dopamine hydrochloride, and 0.1 M sodium chloride). The as-prepared pad was immersed in PBS buffer and DI water to remove the residual reagents.

The drug-releasing pad is further demonstrated in an in vivo application as a self-powered drug-releasing patch (SDP) with sustained release of antibiotics, which is useful for the treatment of wound infection.

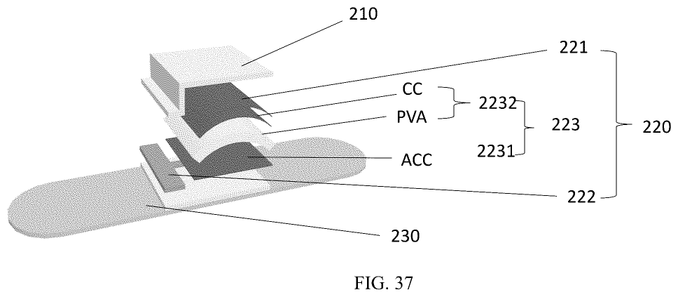

FIG. 37 illustrates schematic of the SDP. As shown, the SDP comprises a substrate 210, a cover pad 230, and a drug-releasing pad 220 arranged between the substrate 210 and the cover pad 230.

The cover pad 230 may contain a PBS solution serving as a electrolyte.

The drug-releasing pad 220 includes a drug-laden layer 221, a printed circuit 222, and a mechanoionic current generator 223.

The drug-laden layer 221 is a polypyrrole (PPy) layer electrodeposited onto a carbon cloth and doped with dopamine and an antibiotic. The printed circuit is an Ag/AgCl printed circuit.

The mechanoionic current generator 223 includes a working electrode 2231 made of an activated carbon cloth (ACC), and a counter electrode 2232 made of a carbon cloth (CC) and a PVA hydrogel.

The counter electrode 2232 may have an arched CC-PVA structure which may be fabricated by the following steps: obtaining a flattened CC-PVA electrode by casting a PVA solution onto CC and freezing; after a first freezing-thawing cycle, putting the CC-PVA electrode into a beaker to introduce the bending deformation; applying another three freezing-thawing cycles to fabricate the arched CC-PVA electrode.

The self-powered anti-bacterial patch may be fabricated by the following steps: preparing a substrate (e.g., a band-aid); printing an Ag/AgCl circuit by coating a layer of Ag/AgCl ink with a mask on a central cotton pad; annealing the printed Ag/AgCl circuit; disposing the mechanoionic current generator 230, the drug-releasing pad 220 and the cover cotton pad 210 onto the substrate layer-by-layer. Finally, fixing the cover cotton pad with a waterproof medical tape.

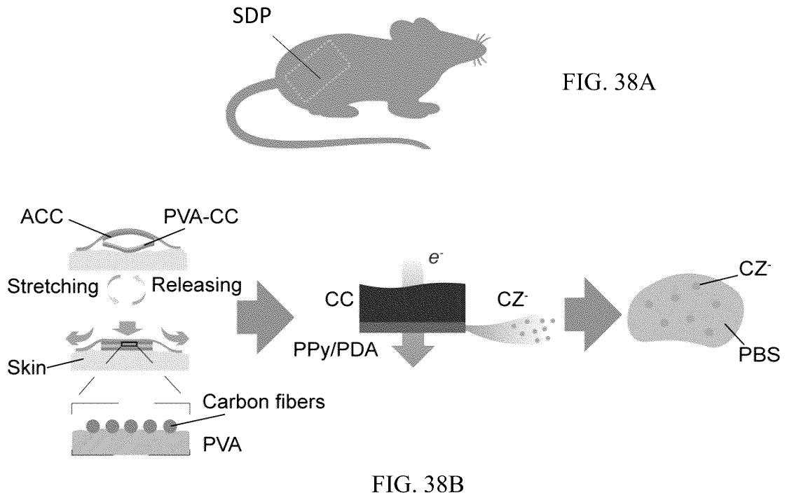

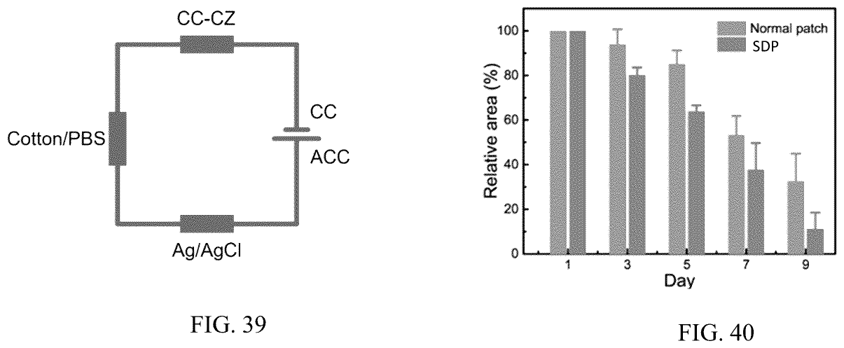

FIG. 38A illustrates a schematic showing application of the SDP on the dorsal flank side of a mouse. A wound incision was generated under the indicated area. FIG. 38B illustrates a schematic image to show the work mechanism of the SDP. FIG. 39 illustrates equivalent circuit of the SDP. The current flows from the ACC electrode, through the Ag/AgCl circuit, the PBS-containing cotton pad, and the drug-laden pad CC-CZ, and back to the CC electrode.

In FIGS. 38A and 38B, the soft and biocompatible components allow the device to cover the wound directly. Movements of the mouse, including eating, breathing, etc., stretches the band-aid and causes the band-aid to be flattened, deforming the arched hydrogel, increasing the contact area with the working electrode. Carbon fibers of the carbon cloth induce the uneven deformation of the PVA hydrogel, generating an electric current. The elastic arched PVA hydrogel recovers its shape after stress relaxation, leading to the separation of ACC and PVA.

A piece of drug-laden pad CC-CZ (1.2 cm×1.4 cm), Pt electrode, and 10 mL PBS buffer formed a two-electrode system. The Pt electrode was connected to a piece of ACC. The CC-CZ was fixed by a Pt clip and connected to a CC-PVA electrode. Hand-tapping the ACC placed on the CC-PVA electrode generated a current pulse and released the drug. After 1 min tapping, 2 mL of the PBS buffer was moved to a quartz cuvette and tested by the UV-Vis spectrometer. Then the PBS buffer was poured back into the two-electrode system and kept for 5 min before the next test. This process was repeated 4 times.

After anesthesia (pentobarbital, 1.5 mg/100 g, intraperitoneal injection), an electric razor and hair removal cream was applied to shave the hair from a dorsal flank region of the mouse. Two symmetrical skin incisions were generated on both sides of each mouse. Then the wounds were infected by 200 μL Staphylococcus aureus (S. aureus) with an initial density of 107 CFU mL 1. After that, all wounds were covered by transparent waterproof wound dressing bandage (Nexcare, 3M). Mice were kept in cages overnight.

The initial areas of wounds were recorded the day after surgery. After anesthesia, a camera (EOS R5, Canon) was used to record the areas of wounds. Then the self-powered anti-bacteria band-aid covered a wound on one side of the mice. Before using, the upper cotton pad was wetted with sterile PBS buffer. A normal band-aid covered the other wound. This process was repeated every two days until the ninth day. The areas of wounds were measured using ImageJ software. After experiments, mice were euthanized by carbondioxide asphyxiation.

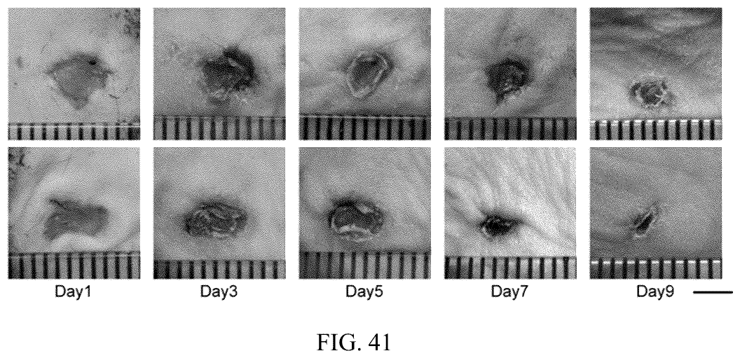

FIG. 40 illustrates the relative area of wounds as a function of healing time. On the last day, the wound area covered by the normal patch and SDP was reduced to 32% and 11% of the initial wound area, respectively. FIG. 41 illustrates photographs of the wound during the healing process with a standard patch (upper) and the SDP (lower).

The functional units and modules of the hydrogel-based current generator in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A mechanoionic current generator, comprising:
a working electrode including an activated carbon cloth;
a counter electrode including a raw carbon cloth and a hydrogel;
wherein the hydrogel has a plurality of flexible and asymmetrically shaped structures; and
the working electrode has a surface immersed in the hydrogel and provided with a plurality of oxygen-containing functional groups.

2. The mechanoionic current generator of claim 1, wherein the plurality of oxygen-containing functional groups is configured to face toward tip portions of the plurality of flexible and asymmetrically shaped structures.

3. The mechanoionic current generator of claim 1, wherein each of the flexible and asymmetrically shaped structures has a pyramid-like shape.

4. The mechanoionic current generator of claim 1, wherein the hydrogel is loaded with mobile ions.

5. The mechanoionic current generator of claim 1, wherein the mobile ions include Li⁺ ions and Cl⁻ ions.

6. A method for fabricating a mechanoionic current generator comprising a working electrode made of activated carbon cloth, a counter electrode made of carbon cloth and a hydrogel, the method comprising:
preparing the working electrode by:
cleaning a piece of carbon cloth;
oxidizing the cleaned carbon cloth in a two-electrode system containing a (NH₄)₂SO₄ aqueous solution; and
reducing the oxidized carbon cloth in a three-electrode system containing a NH₄Cl aqueous solution to form the working electrode; and
preparing the counter electrode by:
pouring a first PVA solution into a mold to obtain a molded PVA;
repeatedly freezing and thawing the molded PVA to obtain a cross-linked hydrogel;
immersing a piece of cleaned carbon cloth into a second PVA solution to obtain a PVA-soaked carbon cloth;
placing the PVA-soaked carbon cloth onto a back side of the cross-linked hydrogel to obtain a combined structure;
repeatedly freezing and thawing the combined structure to obtain a combined electrode; and
keeping the combined electrode in an electrolyte solution for 1 day to load the combined electrode with mobile ions to form the counter electrode.

7. The method according to claim 6, wherein the (NH₄)₂SO₄ aqueous solution has a concentration of 0.1 M and the NH₄Cl aqueous solution has a concentration of 1M.

8. The method according to claim 6, wherein the freezing and thawing comprise freezing the molded PVA at −18° C. for 8 h and thawing the frozen PVA at 25° C. for 3 h.

9. The method according to claim 6, wherein the first PVA solution is a 15 wt % PVA solution and the second PVA solution is 10 wt % PVA solution.

10. A mechanoionic self-powered drug-releasing patch, comprising:
a substrate;
an electrolyte-containing pad; and
a drug-releasing pad disposed between the electrolyte-containing pad and the substrate, comprising:
a drug-laden layer having a surface in contact with the electrolyte-containing pad;
a printed circuit in contact with the drug-laden layer; and
the mechanoionic current generator according to claim 1, wherein, the working electrode is electrically connected to the electrolyte-containing pad through the printed circuit; and the counter electrode is electrically connected to the drug-laden layer.

11. The mechanoionic self-powered drug-releasing patch of claim 10, wherein the plurality of oxygen-containing functional groups is configured to face toward tip portions of the plurality of flexible and asymmetrically shaped structures.

12. The mechanoionic self-powered drug-releasing patch of claim 10, wherein each of the flexible and asymmetrically shaped structures has a pyramid-like shape.

13. The mechanoionic self-powered drug-releasing patch of claim 10, wherein the hydrogel is loaded with mobile ions.

14. The mechanoionic self-powered drug-releasing patch of claim 13, wherein the mobile ions include Li⁺ ions and Cl⁻ ions.

15. The mechanoionic self-powered drug-releasing patch of claim 10, wherein the counter electrode has an arched structure.

16. The mechanoionic self-powered drug-releasing patch of claim 10, wherein the cover pad is a cotton pad containing a PBS solution.

17. The mechanoionic self-powered drug-releasing patch of claim 10, wherein the drug-laden layer is a polypyrrole (PPy) layer electrodeposited onto a carbon cloth and doped with dopamine and an antibiotic.

18. The mechanoionic self-powered drug-releasing patch of claim 10, wherein the printed circuit is an Ag/AgCl printed circuit.
